(19) United States
(12) Patent Application Publication
(10) Pub. No.: US 2022/0299526 A1
(43) Pub. Date: Sep. 22, 2022

(54) CELIAC DISEASE DIAGNOSIS METHOD

(71) Applicant: IRCCS CENTRO NEUROLESI BONINO-PULEJO, Messina (IT)

(72) Inventors: Giuseppe Acri, Dipignano (IT); Placido Bramanti, Messina (IT); Giuseppe Vermiglio, Messina (IT); Alessia Bramanti, Messina (IT); Barbara Testagrossa, Messina (IT); Silvia Marino, Messina (IT); Stefano Costa, Messina (IT); Rosella Ciurleo, Feroleto della Chiesa (IT)

(21) Appl. No.: 17/612,886

(22) PCT Filed: May 25, 2020
(86) PCT No.: PCT/IB2020/054939

§ 371 Date: Nov. 19, 2021

(30) Foreign Application Priority Data
May 24, 2019 (IT) 102019000007214

(51) Int. Cl. G01N 33/68; G01N 21/65

(52) U.S. Cl. CPC G01N 33/6893; G01N 21/65; G01N 2800/24; G01N 2201/06113

(57) ABSTRACT

A diagnosis method for detecting celiac disease comprising the following steps:
a) performing a Raman spectroscopy of a blood serum sample;
b) selecting from the Raman spectrum a first characteristic band of a first indicator of celiac disease, a second characteristic band of a second indicator of celiac disease, and a third characteristic band of a third indicator of celiac disease;
c) performing a deconvolution of at least the second band and the third band of the Raman spectrum, obtaining a respective plurality of Gaussians;
d) calculating a first sum A₂ of the areas of the Gaussians related to the second band, and a second sum A₃ of the areas of the Gaussians related to the third band;
e) calculating a first ratio A₂/A₁ and a second ratio A₃/A₁, wherein A₁ is the area
under the first band;
f) verifying that the first ratio A₂/A₁ is greater than a first threshold value and that the second ratio A₃/A₁ is greater than a second threshold value to confirm the celiac disease diagnosis.

### CROSS-REFERENCE TO RELATED APPLICATIONS

This application claims priority to PCT International Application No. PCT/IB2020/054939 filed on May 25, 2020, which application claims priority to Italian Patent Application No. 102019000007214 filed on May 24, 2019, the disclosures of which are expressly incorporated herein by reference.

### STATEMENT RE: FEDERALLY SPONSORED RESEARCH/DEVELOPMENT

Not applicable.

### BACKGROUND OF THE INVENTION

### Field of the Invention

The present invention relates to a method for diagnosing celiac disease (CD).

### Background Art

Celiac disease (CD) is an immune-mediated systemic disease induced in genetically predisposed subjects by the ingestion of proteins rich in proline and glutamine residues contained in wheat, rye, and barley.

According to the official classification of CD (Marsh-Obehuber), the intestinal damage progresses through various stages up to the final stage of intestinal villous atrophy (Marsh 3), while the initial stage is represented by intraepithelial lymphocyte infiltration (Marsh 1) and crypt hypertrophy (Marsh 2). CD is defined by a very broad clinical spectrum and is associated with an increased risk of morbidity and mortality.

Patients may be absolutely asymptomatic or show even severe clinical symptoms. The spectrum of CD may be divided into 4 main categories.

The classic form, mainly diagnosed during early childhood, is characterized by duodenal atrophy associated with typical intestinal malabsorption symptoms, such as globular abdomen, weight loss, diarrhea, stature-weight retardation, hypoprotidemia.

In the non-classical form, which is continuously increasing, the classic intestinal symptoms are not present, generic intestinal symptoms are often present and extraintestinal signs and symptoms may be present, such as dermatitis, iron-deficiency anemia, hepatitis, cholangitis, hypertransaminasemia, coagulopathy, delayed puberty, osteopenia, arthralgias, aphthous stomatitis, dental enamel defects, alopecia, edema, infertility, depression, cerebellar ataxia.

The potential form is defined by the presence of positive serology, compatible HLA (DQ2 or DQ8) in the absence of duodenal atrophy. Intestinal and extra-intestinal signs and symptoms may or may not be present.

In the silent form, patients are apparently asymptomatic.

CD diagnosis is based on the presence of characteristic lesions in the duodenal biopsy.

Multiple biopsy fragments are required to have a sufficient diagnostic accuracy, and the orientation of the fragments is fundamental for a correct diagnosis. The aforementioned Marsh classification, modified by Oberhuber, is currently used for the histological diagnosis of CD. Partial (Marsh 3A) to total (Marsh 3C) villous atrophy is diagnostic for CD, but only in the presence of a positive serology since similar lesions are found in other pathologies. Marsh stage II, represented by crypt hypertrophy and intraepithelial lymphocyte infiltration, is considered sufficient to diagnose CD in children, in the presence of positive serology, according to what is established by the new ESPGHAN guidelines. Intraepithelial lymphocyte infiltration (IEL), more specifically the presence of more than 25 CD3+ lymphocytes per 100 enterocytes, is less specific for CD and recently other causes of duodenal lymphocytosis have been described and should be considered in the differential diagnosis.

The serological markers usually evaluated for the diagnosis of celiac disease are anti-transglutaminase antibodies (tTG-As) and anti-endomysial antibodies (EMAs). The sensitivity of the EMAs (IgA class) varies in the various studies from 86% to 100% (average 95%) while the specificity stands at values ranging from 90% to 100% (average 99%). For tTG-As (IgA class) sensitivity varies from 61% to 100% (average 85%) and specificity from 86% to 100% (average 95%). Overall, therefore, the combination of EMAs and tTG-As shows good diagnostic accuracy. A new class of antibodies, against deamidated gliadin peptides (DGP-Abs), have shown to be equally accurate compared to EMAs and tTG-As, if not superior in the follow-up for assessing compliance with a gluten-free diet.

However, disadvantageously, in most cases the current background art does not allow a reliable diagnosis of celiac disease to be made in a non-invasive manner, in particular without performing at least one duodenal biopsy.

Although since 2012 the CD diagnosis guidelines of the European Society for Paediatric Gastroenterology Hepatology and Nutrition (ESPGHAN) have introduced the possibility of avoiding duodenal biopsy in pediatric patients, it is also true that this type of diagnostic path is only possible for a minority percentage of patients. In fact, for diagnosing CD without biopsy it is necessary that patients have, at the same time, one of the classic symptoms associated with CD (intestinal malabsorption) and a positivity of the specific serology (anti-transglutaminase antibodies) with a level equal to at least 10 times the normal limit provided for the test. In these patients, confirmation with a further sampling is then required, to determine the anti-endomysial antibodies and HLA (histocompatibility haplotype). In case of positive anti-endomysial antibodies and compatible HLA, the diagnosis may be formalized. Disadvantageously, given the increase in the incidence of CD, considering that all the guidelines for the adult involve biopsy diagnosis and that, both in adults and in children, asymptomatic cases or cases with atypical symptomatology are increasing, it can be deduced that in the near future the number of patients who will have to undergo an endoscopic examination for the biopsy diagnosis of CD will tend to increase rather than decrease.

### SUMMARY OF THE INVENTION

It is an object of the present invention to provide a method for diagnosing celiac disease (CD) which is simple, reliable and automated, and therefore highly efficient, which can always avoid duodenal biopsy for all types of patient, without distinction, even for patients, both children and adults, exhibiting such clinical and serological characteristics as to currently require a biopsy confirmation of the diagnosis.

It is a further object of the present invention to provide an alternative method for diagnosing celiac disease (CD), which is based on Raman spectrophotometric analysis, operating directly on human serum in a non-invasive and non-destructive manner.

The present invention achieves the aforesaid objects by providing a method for diagnosing celiac disease (CD) comprising the following steps:

a) providing as input data a Raman spectrum of a blood serum sample;

b) selecting from said Raman spectrum a first band characteristic of a first indicator of the presence of celiac disease, a second band characteristic of a second indicator of the presence of celiac disease, and a third band characteristic of a third indicator of the presence of celiac disease;

c) possibly performing a deconvolution of at least the second band and the third band of the Raman spectrum, obtaining a respective plurality of Gaussians;

d) possibly calculating a first sum A₂ of the areas of the individual Gaussians related to the second band, and a second sum A₃ of the areas of the individual Gaussians related to the third band;

e) calculating a first ratio A₂/A₁ and a second ratio A₃/A₁, wherein A₁ is the area under the first band of the Raman spectrum;

f) verifying that the first ratio A₂/A₁ is greater than a first threshold value and that the second ratio A₃/A₁ is greater than a second threshold value to confirm that the blood serum sample belongs to a celiac patient.

If the deconvolution is not performed (steps c-d), in step e) A₂ is the area under the second band and A₃ is the area under the third band.

Possibly, the deconvolution is used to recognize peaks within a band, when the latter is noisy, as well as to clean up the tails of the band affected by noise and overlapping. Therefore, performing such a mathematical operation allows to clean the spectrum from the observed noise and to identify the possible presence of other peaks, not visible on the original spectrum. As a result of such a cleaning, the sum of the areas of the individual Gaussians related to the second band and the sum of the areas of the individual Gaussians related to the third band, obtained with the deconvolution performed in the regions of interest, are different and more representative, with respect to the area of the band considered and referable to the original spectrum.

With the aim of identifying a procedural path which exclusively takes into account a physical investigation technique, such as the Raman spectroscopy, a methodology has been developed based on the analysis of the serum of patients by means of the Raman spectrometer.

The new methodology of the present invention has several advantages:
extremely low cost, with respect to the currently used serological techniques, which are based on the ELISA technique, which requires the use of special diagnostic kits;
simplicity of use, experimental repeatability, and speed of execution;
the technique is not invasive;
the technique is not destructive; in fact, the sample in question can be stored and analyzed several times over time, since the only limit of the analysis is given by the degradation of the sample itself.

The method exclusively involves a non-invasive test such as a venipuncture for the collection of serum. It is one of the objectives to add a highly performing diagnostic test to the battery of serological tests with the aim of totally avoiding invasive tests.

The methodology suggested for the diagnosis of Celiac Disease (CD) is based solely on the analysis of human serum by means of the use of Raman spectroscopy and the consequent analysis of the spectrum by calculating the ratio between well-defined areas of the region. Starting from a serum sample, which contains information on the whole human body, and not information attributable to an individual pathology, it was possible to identify three regions, whose areas A₁, A₂ and A₃, taken individually, are not capable of discriminating the patient affected by CD with respect to the healthy one, but, by means of the A₃/A₁ and A₂/A₁ ratios, it is possible to diagnose CD in patients, with at least a 97% reliability proven by statistical analysis.

Further features and advantages of the invention will become apparent in light of the detailed description of some exemplary but not exclusive embodiments.

The dependent claims describe particular embodiments of the invention.

### BRIEF DESCRIPTION OF THE FIGURES

In the description of the invention, reference is made to the accompanying drawings, which are provided by way of non-limiting example, in which:

FIG. 1 shows Raman spectra of a serum sample from a healthy subject (a) and an ill subject (b), respectively;

FIG. 2 shows a first band of the Raman spectra of FIG. 1;

FIG. 3 shows, together, a second band and a third band of the Raman spectra of FIG. 1;

FIG. 4a shows a deconvolution involving the second band of the Raman spectrum of the healthy subject;

FIG. 4b shows a deconvolution involving the second band of the Raman spectrum of the ill subject;

FIG. 5a shows a deconvolution involving the third band of the Raman spectrum of the healthy subject;

FIG. 5b shows a deconvolution involving the third band of the Raman spectrum of the ill subject;

FIG. 6a shows a ROC curve related to a first ratio of areas to measure the accuracy of the diagnostic test;

FIG. 6b shows a ROC curve related to a second ratio of areas to measure the accuracy of the diagnostic test.

### DETAILED DESCRIPTION OF EMBODIMENTS OF THE INVENTION

The method of the invention for diagnosing celiac disease comprises the following steps:

a) providing as input data a Raman spectrum of a blood serum sample;

b) selecting from the Raman spectrum a first band characteristic of a first indicator of the presence of celiac disease, a second band characteristic of a second indicator of the presence of celiac disease, and a third band characteristic of a third indicator of the presence of celiac disease;

c) performing a deconvolution of at least the second band and the third band of the Raman spectrum, obtaining a respective plurality of Gaussians;

d) calculating a first sum A₂ of the areas of the individual Gaussians related to the second band, and a second sum A₃ of the areas of the individual Gaussians related to the third band;

e) calculating a first ratio A₂/A₁ and a second ratio A₃/A₁, wherein A₁ is the area under the first band of the Raman spectrum;

f) verifying that the first ratio A₂/A₁ is greater than a first threshold value and that the second ratio A₃/A₁ is greater than a second threshold value to confirm that the blood serum sample belongs to a celiac patient.

Preferably, between step c) and d) a cleaning of the second band and third band of the spectrum from any possible observed noise is provided.

If this cleaning is performed, the sum A₂ of the areas of the individual Gaussians related to the second band and the sum A₃ of the areas of the individual Gaussians related to the third band, obtained by means of the deconvolution, are different, respectively, from the area A₂′ of the second band and the area A₃′ of the third band of the original Raman spectrum, i.e., of the Raman spectrum of step a).

Preferably, phenylalanine was selected as the first indicator; phospholipids were selected as the second indicator; and amide-I was selected as the third indicator. However, the method of the invention does not exclude the selection of other indicators other than those just indicated.

The first band is within a first sub-range of wavenumbers, preferably between about 1015 cm⁻¹ and 990 cm⁻¹; the second band is within a second sub-range of wavenumbers, preferably between about 1500 cm⁻¹ and 1400 cm⁻¹; and the third band is within a third sub-range of wavenumbers, preferably between about 1750 cm⁻¹ and 1550 cm⁻¹.

In particular, the third band, for example centered near the 1650 cm⁻¹, was assigned to the vibration modes of the amide-I, which mainly involves C═O stretching and, to a lesser extent, C—N stretching, C_{α}—C—N bending vibrations, and N—H bending vibrations in plane of the peptide groups.

The second band, for example centered near the 1450 cm⁻¹, was assigned to the phospholipid vibration modes which involve the bending vibrations of the groups CH₂ and CH₃; while the first band, for example, centered near the 1005 cm⁻¹, was assigned to the phenylalanine vibration mode, which involves the breathing mode of the phenylalanine aromatic ring.

Preferably, the first threshold value and the second threshold value can be defined by performing the following steps:
providing a Raman spectrum of a blood serum sample for each patient of a first known group of celiac patients and for each patient of a second known group of non-celiac patients;
for each patient, both of the first group and of the second group, selecting from the respective Raman spectrum the first band characteristic of the first indicator of the presence of celiac disease, the second band characteristic of the second indicator of the presence of celiac disease, and the third band characteristic of the third indicator of the presence of celiac disease;
performing for each Raman spectrum a deconvolution of at least the second band and the third band, obtaining a respective plurality of Gaussians; calculating the first sum A₂ of the areas of the individual Gaussians related to the second band, and the second sum A₃ of the areas of the individual Gaussians related to the third band; and calculating the first ratio A₂/A₁ and the second ratio A₃/A₁, wherein A₁ is the area under the first band of the Raman spectrum;
performing an analysis of a first ROC curve, obtained by considering the first ratios A₂/A₁ as a database, and determining, in a known manner, the first optimal threshold value using the Youden index, where the aforesaid Youden index is obtained by means of the homonymous function, which depends on sensitivity and specificity, which in turn depend on the considered cut-off value. The cut-off value at which the Youden index is maximum therefore represents the optimal cut-off value;
performing an analysis of a second ROC curve, obtained by considering the second ratios A₃/A₁ as the database, and determining, similarly to what was described above, the second optimal threshold value using the Youden index.

As is known, most diagnostic tests produce a quantitative result. To discriminate between healthy and ill people it is necessary to have a threshold or cut-off value. In an ideal situation, healthy and ill people return different test values and the cut-off value is immediately determined. In real situations, there is always some overlap in the distribution of healthy and ill people. Sensitivity and specificity are inversely related in relation to the selection of the cut-off. The adoption of a threshold which offers high sensitivity leads to a loss of specificity and vice versa.

In the proposed method, although the distribution of healthy and ill people is distinct enough, to obtain the threshold values which minimize the probability of finding false positives and false negatives, it is preferable to construct two ROC (Receiver Operating Characteristic) curves in a known manner, with sensitivity on the ordinates and (1-specificity) on the abscissas, considering the first ratios A₂/A₁ and the second ratios A₃/A₁, respectively, as the database, therefore obtaining the first optimal threshold value and the second optimal threshold value.

It was found that the second threshold value, related to the ratios A₃/A₁, is greater than the first threshold value, related to the ratios A₂/A₁.

The information obtained from a Raman scattering experiment is graphically depicted as a diagram (Raman spectrum) where the abscissas report the Raman shifts corresponding to the energy jumps between the fundamental vibrational levels and expressed in cm⁻¹ (wavenumber, keeping in mind the direct proportionality between the energy and the inverse of the wavelength of an electromagnetic radiation). Raman intensities proportional to the number of Stokes photons collected by the instrument detector are shown on the ordinates. The range of energies reported in a normal Raman spectrum can extend from a few tens of cm⁻¹ up to about 3500 cm⁻¹, a region in which almost all the fundamental molecular vibrations fall.

In particular, the Raman spectrum of the blood serum sample can be acquired in a range of wavenumbers between 3500 cm⁻¹ and 300 cm⁻¹, preferably between 3300 cm⁻¹ and 400 cm⁻¹, even more preferably between 2500 cm⁻¹ and 500 cm⁻¹.

Raman spectroscopy can be performed by means of any Raman spectrometer having a laser source, such as, for example, a diode laser source, with a wavelength preferably, but not necessarily, equal to 780 nm.

The spectrometer is connected to a detector, preferably a charge-coupled device (CCD), possibly integrated with a monochromator.

The Raman spectrum can, for example, be acquired by performing a number of scans of the serum sample between 25 and 35, with an exposure time between 30 and 90 seconds for each scan.

Preferably, in step c) of the method of the invention, the parameter selected to perform the deconvolution, in addition to the respective band of the Raman spectrum, is the full width at half maximum (FWHM) of the respective band, expressed in wavenumbers.

An experimental work is described below, which allowed developing the method of the invention.

Serum samples from healthy subjects, not affected by celiac disease, and from celiac subjects, with diagnosis proven by duodenal biopsy, have been analyzed. In total, 263 patients have been analyzed, including 21 adults and 242 children (3-16 years).

The serum samples were analyzed, 3-4 hours after the serum sampling, by means of a DXR Smart Raman spectrometer, which has a diode laser with a wavelength of 780 nm as source.

The spectra were acquired in a range of wavenumbers between 3300 cm⁻¹ and 400 cm⁻¹, performing 32 scans of the serum sample, with an exposure time of 60 seconds for each scan.

The laser was used at the maximum power provided by the system (24 mW) at the output of a 50 μm pinhole opening.

The detector connected to the DXR Smart Raman is a charge-coupled device (CCD) which, in addition to having very high sensitivity, allows to simultaneously study a broad spectral band, instead of a single wavelength at a time. In particular, the use of a CCD integrated with a monochromator allows to obtain, in one go, the entire Raman spectrum with advantages in terms of experimental simplicity and speed of execution of the experiment.

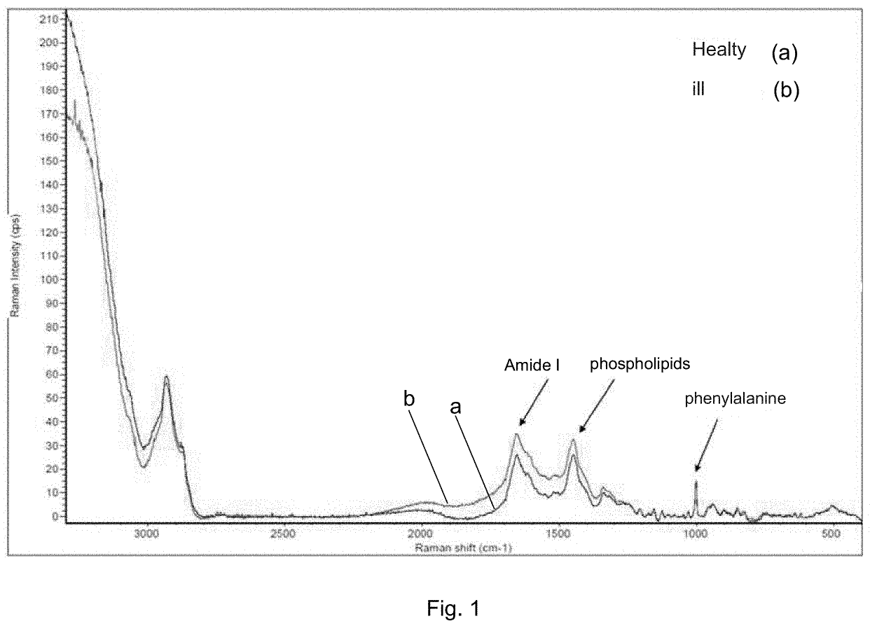

As an example, the spectra of a healthy subject and a celiac subject are shown in the Figures. The spectra obtained have a typical trend depicted in FIG. 1, where the Raman spectra (3300 cm⁻¹-400 cm⁻¹) of a healthy subject (a) and of a subject with celiac disease (b) are shown. FIG. 1 also highlights the bands of interest for the present study. In this case, the following were considered as indicators of the presence of celiac disease:
phenylalanine, preferably the breathing vibration mode of the phenylalanine aromatic ring, corresponding to the band between 1015 cm⁻¹ and 990 cm⁻¹;
phospholipids, preferably the phospholipid vibration modes, corresponding to the band between 1500 cm⁻¹ and 1400 cm⁻¹;
amide-I, preferably the amide-I vibration modes, corresponding to the band between 1750 cm⁻¹ and 1550 cm⁻¹.

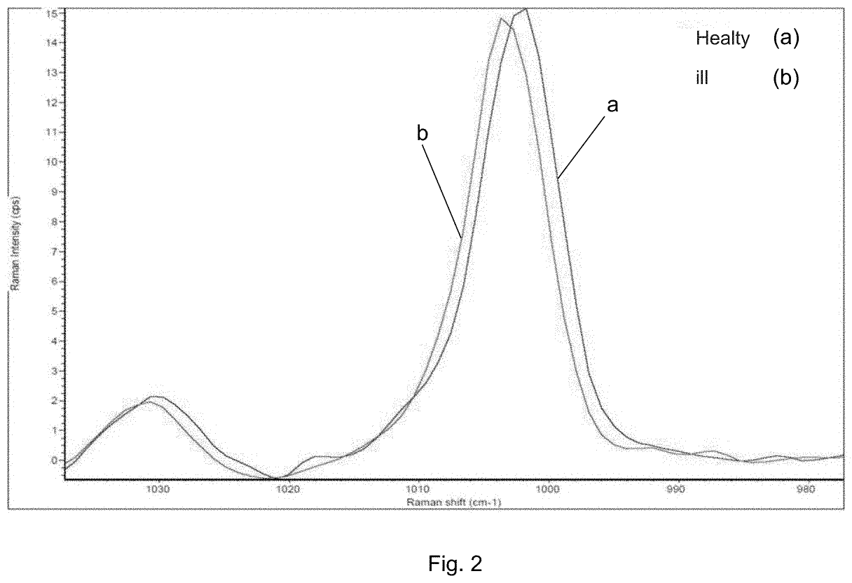

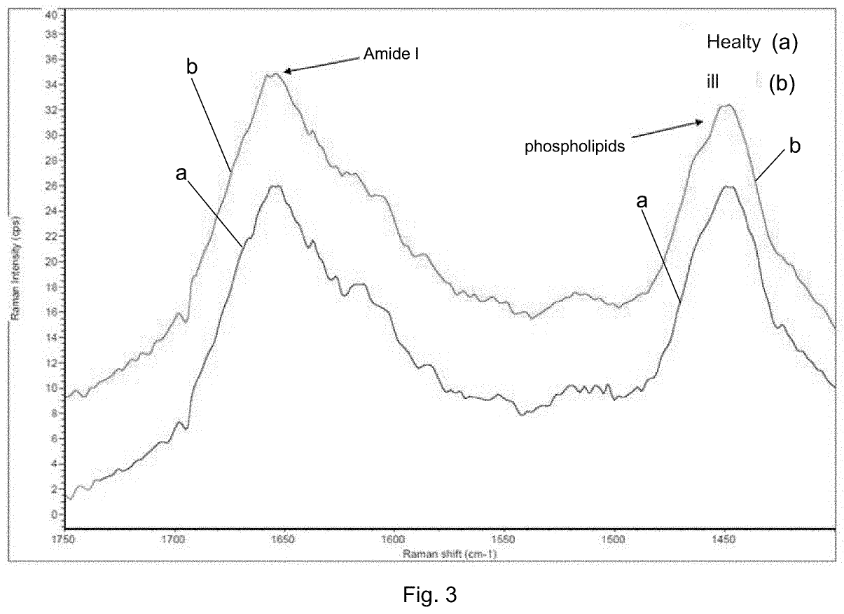

FIGS. 2 and 3 better highlight, in detail, the regions of interest, always with reference to the comparison between healthy subject (a) and ill subject (b).

It has been found that, while the band relating to the breathing vibration mode, or simply breathing mode, of the phenylalanine aromatic ring has a substantially indistinguishable peak between healthy and ill subjects, the other two bands have a fairly variable trend. For example, for some healthy subjects, the Raman intensity is lower than that of ill subjects, while for other healthy subjects the Raman intensity is greater than that of ill subjects.

It was therefore decided to develop a procedure for the identification of subjects suffering from celiac disease based on the relationship between the bands of interest. The band related to the breathing vibration mode of the phenylalanine aromatic ring has been used to normalize the spectra. Such a region, in fact, is not sensitive to the changes in the conformation of proteins, and is therefore used to normalize the Raman spectra of proteins.

In particular, the following two bands can be deconvolved:
the band between 1500 cm⁻¹ and 1400 cm⁻¹;
and the band between 1750 cm⁻¹ and 1550 cm⁻¹;

Deconvolution is a well-known mathematical operation which allows resolving overlapping or very close bands. The parameters selected to perform the above operation are:
the shape of the respective band;
the full width at half maximum of the band (FWHM), expressed in wavenumbers.

A Gaussian fit with high sensitivity was chosen to be performed and, for the 1500 cm⁻¹-1400 cm⁻¹ and 1750 cm⁻¹-1550 cm⁻¹ bands, an FWHM equal to 20.

The spectrum, in such regions, was therefore deconvolved and, once the noise possibly observed was cleaned, the areas of all the Gaussians present therein were added (these sums of areas being called A₂ and A₃).

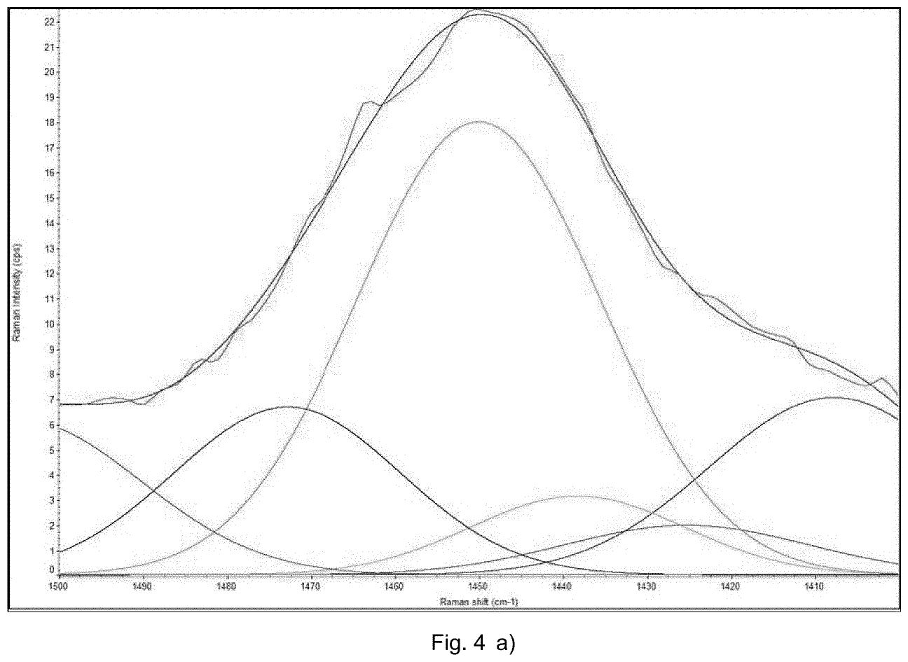

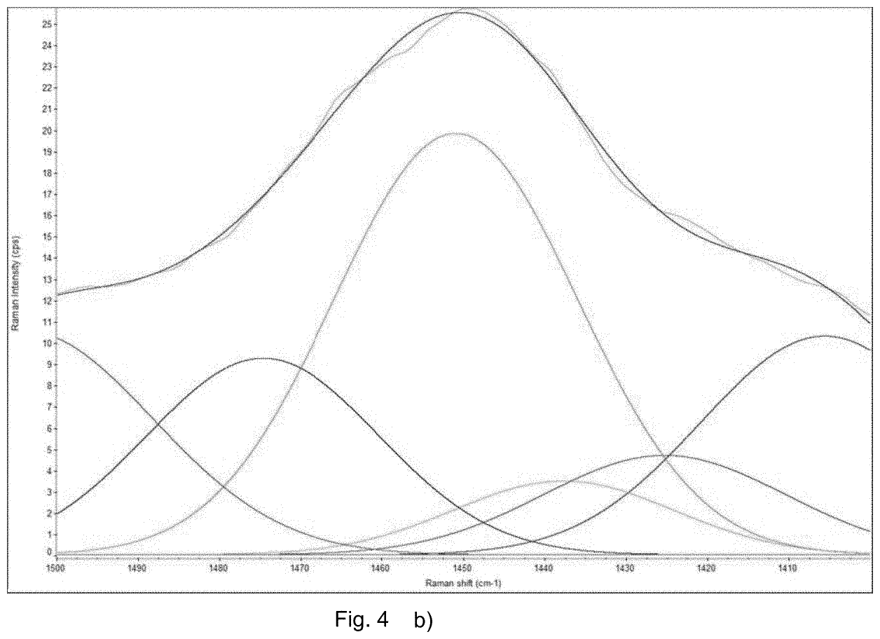

FIG. 4 shows, by way of example, the deconvolved spectra, by means of Gaussian functions, in the range between 1500 cm⁻¹ and 1400 cm⁻¹ of a healthy subject (a) and of an ill subject (b), respectively.

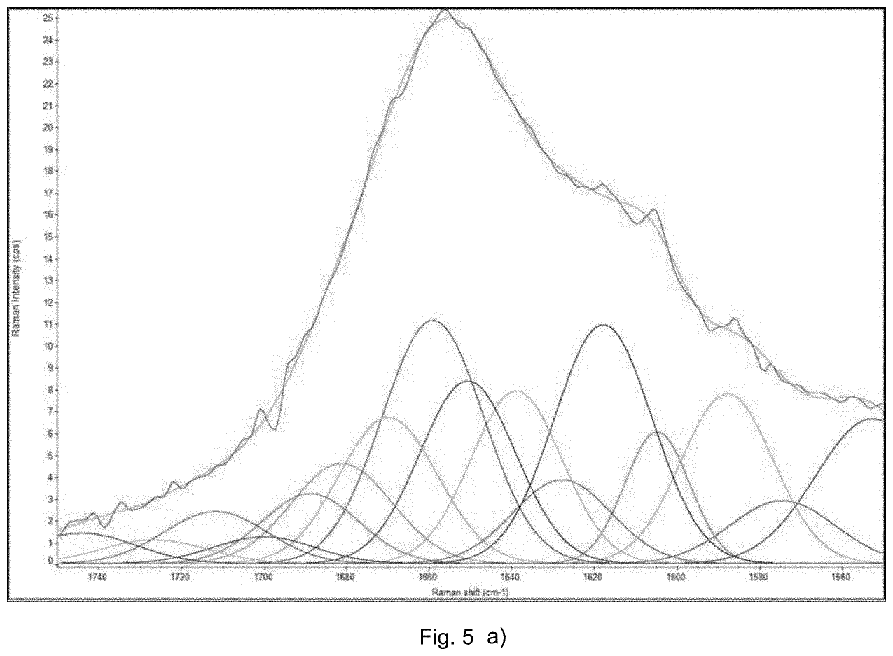

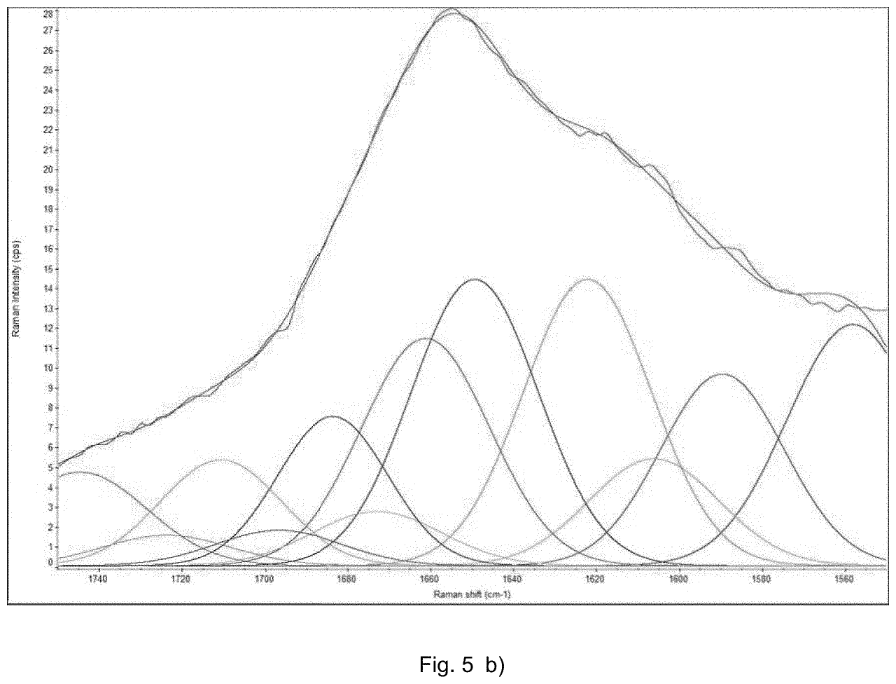

FIG. 5 shows the deconvolved spectra, by means of Gaussian functions, in the range between 1750 cm⁻¹ and 1550 cm⁻¹ of a healthy subject (a) and of an ill subject, respectively.

Such sums of areas A₂ and A₃ were therefore compared to area A₁, under the peak related to the breathing vibration mode of the phenylalanine aromatic ring and calculated in the range between 1015 cm⁻¹ and 990 cm⁻¹.

Such A₂/A₁ and A₃/A₁ ratios return significantly different values between celiac and non-celiac patients.

Table 1 shows the comparison between a healthy subject and a subject affected by celiac disease.

Such a comparison showed that the A₂/A₁ and A₃/A₁ ratios are greater in the case of the celiac subject. By repeating the procedure on 261 other subjects, ill and healthy, the same behavior was found.

TABLE 1

| Areas Ratio | healthy patient | ill patient |
|---|---|---|
| A₂/A₁ | 13.38 | 31.85 |
| A₃/A₁ | 24.61 | 57.38 |

The analysis of the ROC (Receiver Operating Characteristic) curve was therefore used to measure the accuracy of the diagnostic test along the whole range of possible values.

The area under the ROC curve, also called AUC (Area Under the Curve), defines the diagnostic accuracy of the test and a diagnostic test with AUC having a value ≥80% is considered accurate.

For the interpretation of the values of the area under the ROC curve, the classification suggested by Swets is used, i.e.:

| | |
|---|---|
| AUC = 0.5 | non-informative test |
| 0.5 < AUC ≤ 0.7 | inaccurate test |
| 0.7 < AUC ≤ 0.9 | moderately accurate test |
| 0.9 < AUC < 1.0 | highly accurate test |
| AUC = 1.0 | perfect test. |

In order to identify the optimal threshold value for the A₂/A₁ and A₃/A₁ ratios (the so-called best cut-off value), i.e., the value of the test which maximizes the difference between the true positives (i.e., the proportion of individuals who have a value of the test altered, among all those really affected by the disease) and the false positives (i.e. the proportion of individuals who, despite having a value of the test altered, are not affected by the disease), the Youden index was used.

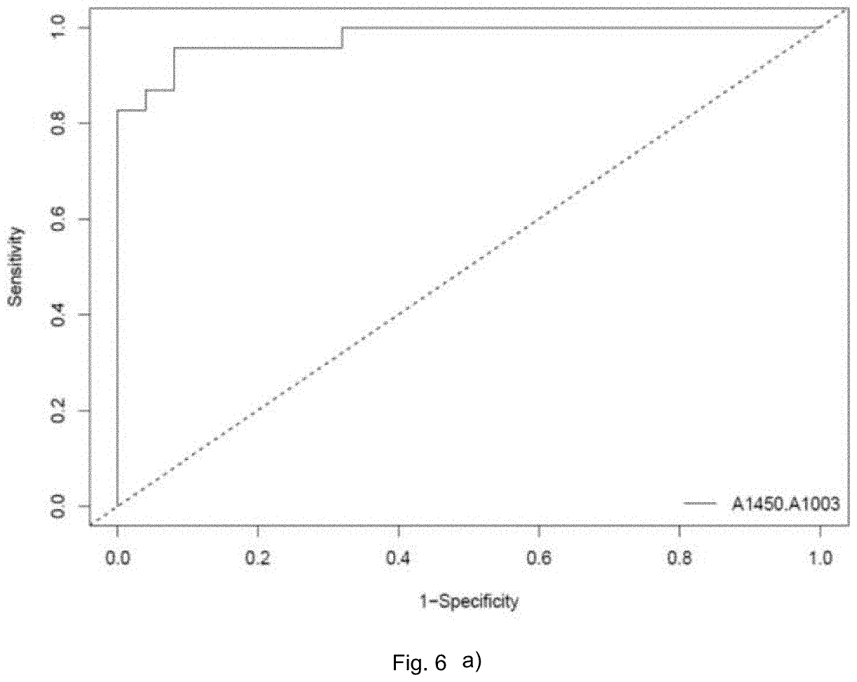

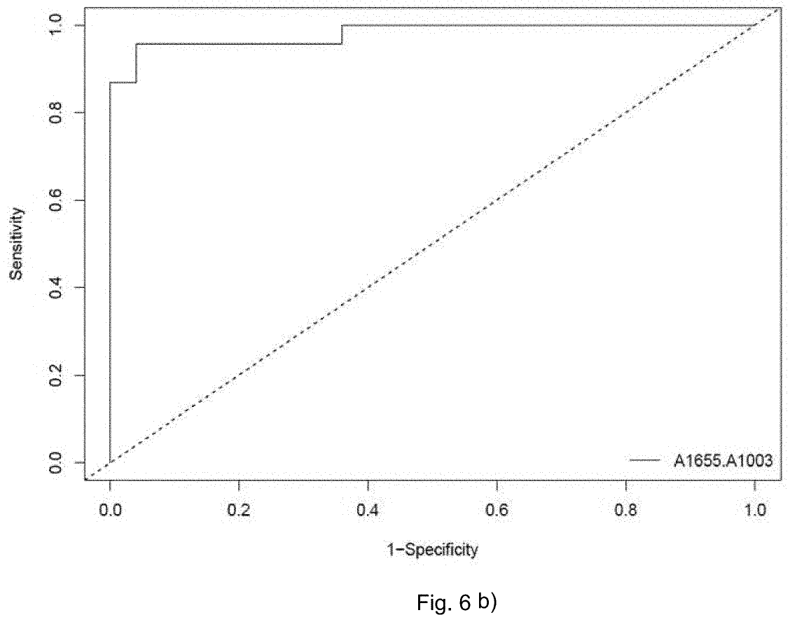

FIGS. 6a and 6b show the ROC curves related to the A₂/A₁ and A₃/A₁ ratios, respectively, while in Table 2 the cut-off values obtained from the analysis of these curves are indicated.

TABLE 2

| Areas Ratio | Cut-off |
|---|---|
| A₂/A₁ | 21.97 |
| A₃/A₁ | 40.14 |

By analyzing the serum of the 263 patients in the manner described above, an A₂/A₁ ratio lower than 21.97 and an A₃/A₁ ratio lower than 40.14 can be used as a discriminator for healthy subjects.

Advantageously, both ratios are indicative of the presence of celiac disease, as shown by the statistical tests reported below.

In fact, such threshold values were chosen as reference values and, on this basis, the diagnostic efficiency test was performed, calculating sensitivity (i.e., the ability to correctly identify the ill subjects) and diagnostic specificity (i.e., the ability to correctly identify the healthy subjects) of the new method.

The results obtained were reported in Tables 3 and 4, related to the A₂/A₁ and A₃/A₁ ratios, respectively.

The same tables also indicate the lower limit and the upper limit of sensitivity and specificity, calculated taking into account a 95% confidence interval.

TABLE 3
Sensitivity and Specificity related to the ratio A₂/A₁

| | Estimate | Lower limit | Upper limit |
|---|---|---|---|
| A₂/A₁ | (%) | (95%) | (95%) |
| Sensitivity | 95.7 | 78.1 | 99.9 |
| Specificity | 92.0 | 74.0 | 99.0 |

TABLE 4
Sensitivity and specificity related to the ratio A₃/A₁

| | Estimate | Lower limit | Upper limit |
|---|---|---|---|
| A₃/A₁ | (%) | (95%) | (95%) |
| Sensitivity | 95.7 | 78.1 | 99.9 |
| Specificity | 96.0 | 79.6 | 99.9 |

Table 5 shows the AUC values calculated from the ROC curves identified in FIGS. 6a and 6b, as well as the lower limit and the upper limit, calculated taking into account a 95% confidence interval. In both cases the calculated AUC value is greater than 0.9 and this, in fact, makes the test highly accurate.

TABLE 5
AUC values obtained and relative limits

| Areas | | Lower limit | Upper limit |
|---|---|---|---|
| Ratio | AUC | (95%) | (95%) |
| A₂/A₁ | 0.97739 | 0.94436 | 1.0 |
| A₃/A₁ | 0.98087 | 0.94783 | 1.0 |

Similar surprising results have been obtained with a further embodiment of the method of the invention. Such a method for diagnosing celiac disease comprises, or consists of, the following steps:

a) providing as input data a Raman spectrum of a blood serum sample;

b) selecting from the Raman spectrum a first band characteristic of a first indicator of the presence of celiac disease, a second band characteristic of a second indicator of the presence of celiac disease, and a third band characteristic of a third indicator of the presence of celiac disease;

c) calculating a first ratio A₂/A₁ and a second ratio A₃/A₁, wherein A₁ is the area under the first band, A₂ is the area under the second band, and A₃ is the area under the third band;

d) verifying that the first ratio A₂/A₁ is greater than a first threshold value and that the second ratio A₃/A₁ is greater than a second threshold value to confirm that the blood serum sample belongs to a celiac patient.

Preferably, phenylalanine was selected as the first indicator; phospholipids were selected as the second indicator; and amide-I was selected as the third indicator. However, the method of the invention does not exclude the selection of other indicators other than those just indicated.

The first band is within a first sub-range of wavenumbers, preferably between about 1015 cm⁻¹ and 990 cm⁻¹; the second band is within a second sub-range of wavenumbers, preferably between about 1500 cm⁻¹ and 1400 cm⁻¹; and the third band is within a third sub-range of wavenumbers, preferably between about 1750 cm⁻¹ and 1550 cm⁻¹.

In particular, the third band, for example centered near the 1650 cm⁻¹, was assigned to the vibration modes of the amide-I, which mainly involves C═O stretching and, to a lesser extent, C—N stretching, C_{α}—C—N bending vibrations, and N—H bending vibrations in plane of the peptide groups.

The second band, for example centered near the 1450 cm⁻¹, was assigned to the phospholipid vibration modes which involve the bending vibrations of the groups CH₂ and CH₃; while the first band, for example, centered near the 1005 cm⁻¹, was assigned to the phenylalanine vibration mode, which involves the breathing of the phenylalanine aromatic ring.

Preferably, the first threshold value and the second threshold value can be defined by performing the following steps:
providing a Raman spectrum of a blood serum sample for each patient of a first known group of celiac patients and for each patient of a second known group of non-celiac patients;
for each patient, both of the first group and of the second group, selecting from the respective Raman spectrum the first band characteristic of the first indicator of the presence of celiac disease, the second band characteristic of the second indicator of the presence of celiac disease, and the third band characteristic of the third indicator of the presence of celiac disease;
for each Raman spectrum, calculating the first ratio A₂/A₁ and the second ratio A₃/A₁, where A₁ is the area under the first band, A₂ is the area under the second band, and A₃ is the area under the third band;
performing an analysis of a first ROC curve, obtained by considering the first ratios A₂/A₁ as a database, and determining, in a known manner, the first optimal threshold value using the Youden index, where the aforesaid Youden index is obtained by means of the homonymous function, which depends on sensitivity and specificity, which in turn depend on the considered cut-off value. The cut-off value at which the Youden index is maximum therefore represents the optimal cut-off value;
performing an analysis of a second ROC curve, obtained by considering the second ratios A₃/A₁ as the database, and determining, similarly to what was described above, the second optimal threshold value using the Youden index.

As is known, most diagnostic tests produce a quantitative result. To discriminate between healthy and ill people it is necessary to have a threshold or cut-off value. In an ideal situation, healthy and ill people return different test values and the cut-off value is immediately determined. In real situations, there is always some overlap in the distribution of healthy and ill people. Sensitivity and specificity are inversely related in relation to the selection of the cut-off. The adoption of a threshold which offers high sensitivity leads to a loss of specificity and vice versa.

In the proposed method, although the distribution of healthy and ill people is distinct enough, to obtain the threshold values which minimize the probability of finding false positives and false negatives, it is preferable to construct two ROC (Receiver Operating Characteristic) curves in a known manner, with sensitivity on the ordinates and (1-specificity) on the abscissas, considering the first ratios A₂/A₁ and the second ratios A₃/A₁, respectively, as the database, therefore obtaining the first optimal threshold value and the second optimal threshold value.

It was found that the second threshold value, related to the A₃/A₁ ratios, is greater than the first threshold value, related to the A₂/A₁ ratios.

1. A diagnosis method for detecting celiac disease comprising the following steps:
a) providing as input data a Raman spectrum of a blood serum sample;
b) selecting from said Raman spectrum a first characteristic band of a first indicator of the presence of celiac disease, a second characteristic band of a second indicator of the presence of celiac disease, and a third characteristic band of a third indicator of the presence of celiac disease;
c) performing a deconvolution of at least the second band and the third band of the Raman spectrum, obtaining a respective plurality of Gaussians;
d) calculating a first sum A₂ of the areas of the individual Gaussians related to the second band, and a second sum A₃ of the areas of the individual Gaussians related to the third band;
e) calculating a first ratio A₂/A₁ and a second ratio A₃/A₁, wherein A₁ is the area under the first band of the Raman spectrum;
f) verifying that the first ratio A₂/A₁ is greater than a first threshold value and that the second ratio A₃/A₁ is greater than a second threshold value to confirm that the blood serum sample belongs to a celiac patient.

2. The method according to claim 1, wherein said first indicator is given by phenylalanine; wherein said second indicator is given by phospholipids; and wherein said third indicator is given by amide-I.

3. The method according to claim 1, wherein said first indicator is given by the breathing vibration mode of the phenylalanine aromatic ring; wherein said second indicator is given by the phospholipid vibration modes; and wherein said third indicator is given by the amide-I vibration modes.

4. The method according to claim 1, wherein the first band is comprised in a first sub-range of wavenumbers, the second band is comprised in a second sub-range of wavenumbers, and the third band is comprised in a third sub-range of wavenumbers.

5. The method according to claim 4, wherein said first sub-range of wavenumbers is between about 1015 cm⁻¹ and 990 cm⁻¹, said second sub-range of wavenumbers is between about 1500 cm⁻¹ and 1400 cm⁻¹, and said third sub-range of wavenumbers is between about 1750 cm⁻¹ and 1550 cm⁻¹.

6. The method according to claim 1, wherein said first threshold value and said second threshold value can be defined by performing the following steps:
providing a Raman spectrum of a blood serum sample for each patient of a first known group of celiac patients and for each patient of a second known group of non-celiac patients;
for each patient, both of the first group and of the second group, selecting from the respective Raman spectrum the first band characteristic of the first indicator of the presence of celiac disease, the second band characteristic of the second indicator of the presence of celiac disease, and the third band characteristic of the third indicator of the presence of celiac disease;
performing for each Raman spectrum a deconvolution of at least the second band and the third band, obtaining a respective plurality of Gaussians; calculating the first sum A₂ of the areas of the individual Gaussians related to the second band, and the second sum A₃ of the areas of the individual Gaussians related to the third band; and calculating the first ratio A₂/A₁ and the second ratio A₃/A₁, wherein A₁ is the area under the first band of the Raman spectrum;
performing an analysis of a first ROC curve considering as database the first ratios A₂/A₁ and calculating the first threshold value by means of the Youden's index; and performing an analysis of a second ROC curve considering as database the second ratios A₃/A₁ and calculating the second threshold value by means of Youden's index.

7. The method according to claim 1, wherein said second threshold value is greater than said first threshold value.

8. The method according to claim 1, wherein the Raman spectrum is acquired in a range of wavenumbers between 3500 cm⁻¹ and 300 cm⁻¹.

9. The method according to al y claim 1, wherein, in order to obtain the Raman spectrum of the blood serum sample, a Raman spectrometer is used.

10. The method according to claim 9, wherein a detector is connected to said Raman spectrometer.

11. The method according to claim 9 or 10, wherein the Raman spectrum is acquired by performing a number of scans of the serum sample between 25 and 35, with an exposure time between 30 and 90 seconds for each scan.

12. The method according to claim 1, wherein in step c) parameter selected to perform the deconvolution, in addition to the respective band of the Raman spectrum, is the full width at half maximum (FWHM) of the respective band, expressed in wavenumbers.

13. The method according to claim 8, wherein the Raman spectrum is acquired in a range of wavenumbers between 3300 cm⁻¹ and 400 cm⁻¹.

14. The method according to claim 13, wherein the Raman spectrum is acquired in a range of wavenumbers between 2500 cm⁻¹ and 500 cm⁻¹.

15. The method according to claim 10, wherein said detector is a charge-coupled device (CCD).

16. The method according to claim 15, wherein said detector is integrated with a monochromator.

17. The method according to claim 1, wherein between step c) and d) a cleaning of the second band and third band of the spectrum from any possible observed noise signal is provided.

18. A diagnosis method for detecting celiac disease comprising the following steps:
a) providing as input data a Raman spectrum of a blood serum sample;
b) selecting from the Raman spectrum a first band characteristic of a first indicator of presence of celiac disease, a second band characteristic of a second indicator of presence of celiac disease, and a third band characteristic of a third indicator of presence of celiac disease;
c) calculating a first ratio A₂/A₁ and a second ratio A₃/A₁, wherein A₁ is the area under the first band, A₂ is the area under the second band, and A₃ is the area under the third band;
d) verifying that the first ratio A₂/A₁ is greater than a first threshold value and that the second ratio A₃/A₁ is greater than a second threshold value to confirm that the blood serum sample belongs to a celiac patient.
